(19) United States
(12) Patent Application Publication
(10) Pub. No.: US 2017/0049469 A1
(43) Pub. Date: Feb. 23, 2017

(54) SYSTEM AND METHOD FOR TREATING HEPATIC AREA VEIN OCCLUSION

(71) Applicant: Mao Heng Zu, Xuzhou (CN)

(72) Inventor: Mao Heng Zu, Xuzhou (CN)

(21) Appl. No.: 14/833,026

(22) Filed: Aug. 21, 2015

(51) Int. Cl. A61B 17/3207; A61B 34/00; A61B 34/30; A61B 6/00; A61B 90/00

(52) U.S. Cl. CPC A61B 17/3207; A61B 6/485; A61B 6/504; A61B 90/37; A61B 34/30; A61B 6/5211; A61B 34/25; A61B 2034/303; A61B 2034/252; A61B 2090/374; A61B 2090/376

(57) ABSTRACT

A system and a method are disclosed for treating hepatic area vein occlusion problem. There are five major techniques, used in combined manner or separately, showing effective cure rate. The first technique is to use multiple types of guiding wire through-to-through skills. The second technique is to use one or more accessory hepatic veins (AHV) to drain hepatic blood into inferior vena cava for decreasing pressure inside the liver. The third technique is to puncture through occlusion of veins. The fourth technique is reverse direction membrane puncture. The fifth technique is thrombosis handling.

### FIELD OF INVENTION

The present invention is related to system and method for treating vein occlusion and more particularly related to system and method for treating vein occlusion in hepatic area.

### BACKGROUND

The circulatory system is a critical part of human body. The circulatory system is also called the cardiovascular system, which is an organ system that permits blood to circulate and transport nutrients (such as amino acids and electrolytes), oxygen, carbon dioxide, hormones, and blood cells to and from the cells in the body to provide nourishment and help in fighting diseases, stabilize temperature and pH, and maintain homeostasis. The study of the blood flow is called hemodynamics. The study of the properties of the blood flow is called hemorheology.

Blood vessels are the part of the circulatory system that transports the blood throughout the human body. There are three major types of blood vessels. Arteries carry the blood away from the heart. Capillaries enable actual exchange of water and chemicals between the blood and the tissues. Veins carry blood from the capillaries back toward the heart.

The circulatory system includes the pulmonary circulation, a “loop” through the lungs where blood is oxygenated; and the systemic circulation, a “loop” through the rest of the body to provide oxygenated blood. The systemic circulation can also be seen to function in two parts—a macrocirculation and a microcirculation. An average adult contains five to six quarts (roughly 4.7 to 5.7 liters) of blood, accounting for approximately 7% of their total body weight. Blood consists of plasma, red blood cells, white blood cells, and platelets.

In addition to the pulmonary circulation for oxygen and carbon oxide exchange, the circulatory system works with the digestive system to provide the nutrients the system needs to keep the heart pumping. A major interaction between the circulatory system and the digestive system occurs in the liver. The blood supply of the liver is unique among all organs of the body due to the hepatic portal vein system. Blood traveling to the spleen, stomach, pancreas, gallbladder, and intestines passes through capillaries in these organs and is collected into the hepatic portal vein. The hepatic portal vein then delivers this blood to the tissues of the liver where the contents of the blood are divided up into smaller vessels and processed before being passed on to the rest of the body. Blood leaving the tissues of the liver collects into the hepatic veins that lead to the vena cava and return to the heart. The liver also has its own system of arteries and arterioles that provide oxygenated blood to its tissues just like any other organ.

From the histology aspect, the study of microscopic anatomy shows two major types of liver cell: parenchymal cells and non-parenchymal cells. 70-85% of the liver volume is occupied by parenchymal hepatocytes.

Non-parenchymal cells constitute 40% of the total number of liver cells but only 6.5% of its volume. The liver sinusoids are lined with two types of cell, sinusoidal endothelial cells, and phagocytic Kupffer cells. Hepatic stellate cells are some of the non-parenchymal cells that are external to the sinusoid in the space of Disse. Each of the lobes is seen to be made up of hepatic lobules; a vein goes from the center, which then joins to the hepatic vein to carry blood out from the liver. On the surface of the lobules, there are ducts, veins and arteries that carry fluids to and from them. A distinctive component of a lobule is the portal triad.

From the functional anatomy aspect, the central area of the liver, where the common bile duct, hepatic portal vein, and the hepatic artery proper enter is the hilum known as the porta hepatis (gateway to the liver) or the transverse fissure of the liver. The duct, vein, and artery divide into left and right branches, and the areas of the liver supplied by these branches constitute the functional left and right lobes. The functional lobes are separated by the imaginary plane, Cantlie's line joining the gallbladder fossa to the inferior vena cava. The plane separates the liver into the true right and left lobes. The middle hepatic vein also demarcates the true right and left lobes. The right lobe is further divided into an anterior and posterior segment by the right hepatic vein. The left lobe is divided into the medial and lateral segments by the left hepatic vein.

The liver gets a dual blood supply from the hepatic portal vein and hepatic arteries. Supplying approximately 75% of the liver's blood supply, the hepatic portal vein carries venous blood drained from the spleen, gastrointestinal tract, and its associated organs. The hepatic arteries supply arterial blood to the liver, accounting for the remainder of its blood flow. Oxygen is provided from both sources; approximately half of the liver's oxygen demand is met by the hepatic portal vein, and half is met by the hepatic arteries.

Blood flows through the liver sinusoids and empties into the central vein of each lobule. The central veins coalesce into hepatic veins, which leave the liver.

The liver has a wide range of functions, including detoxification of various metabolites, protein synthesis, and the production of biochemicals necessary for digestion. To keep these important functions normally, blood transmission in the liver needs to be performed successfully. When vascular occlusion occurs, no matter in arteries, veins or capillaries, the vascular pressure may rise abnormally. Furthermore, liver cells may get damaged due to vascular occlusion.

Vascular occlusion is a blockage of a blood vessel, usually with a clot. It differs from thrombosis in that it can be used to describe any form of blockage, not just one formed by a clot. When it occurs in a major vein, it can, in some cases, cause deep vein thrombosis. The condition is also relatively common in the retina, and can cause partial or total loss of vision. An occlusion can often be diagnosed using Doppler sonography (a form of ultrasound).

Budd-Chiari Syndrome (BCS), which is named after George Budd, a British physician, and Hans Chiari, an Austrian pathologist, is a condition caused by occlusion of the hepatic veins that drain the liver. It presents with the classical triad of abdominal pain, ascites, and liver enlargement. The formation of a blood clot within the hepatic veins can lead to Budd-Chiari syndrome. The syndrome can be fulminant, acute, chronic, or asymptomatic.

The acute syndrome presents with rapidly progressive severe upper abdominal pain, yellow discoloration of the skin and whites of the eyes, liver enlargement, enlargement of the spleen fluid accumulation within the peritoneal cavity, elevated liver enzymes, and eventually encephalopathy. The fulminant syndrome presents early with encephalopathy and ascites. Liver cell death and severe lactic acidosis may be present as well. Caudate lobe enlargement is often present. The majority of patients have a slower-onset form of Budd-Chiari syndrome. This can be painless. A system of venous collaterals may form around the occlusion which may be seen on imaging as a “spider's web.” Patients may progress to cirrhosis and show the signs of liver failure.

On the other hand, incidental finding of a silent, asymptomatic form may not be a cause for concern.

The cause for the disease cannot be found in about half of the patients. About 75% of Budd-Chiari Syndrome is caused by thrombosis of the hepatic vein. About 25% Budd-Chiari Syndrome (25%) is caused by compression of the hepatic vein by an outside structure (e.g. a tumor). Hepatic vein thrombosis is associated with the following in decreasing order of frequency: polycythemia vera, pregnancy, postpartum state, use of oral contraceptives, paroxysmal nocturnal hemoglobinuria, hepatocellular carcinoma, and lupus anticoagulants.

Budd-Chiari syndrome is also seen in Infection such as tuberculosis, congenital venous webs and occasionally in inferior vena caval stenosis.

Often, the patient is known to have a tendency towards thrombosis, although Budd-Chiari syndrome can also be the first symptom of such a tendency. Examples of genetic tendencies include protein C deficiency, protein S deficiency, the Factor V Leiden mutation, hereditary anti-thrombin deficiency and prothrombin mutation G20210A. An important non-genetic risk factor is the use of estrogen-containing (combined) forms of hormonal contraception. Other risk factors include the antiphospholipid syndrome, aspergillosis, Behçet's disease, dacarbazine, pregnancy, and trauma.

Many patients have Budd-Chiari syndrome as a complication of polycythemia vera (myeloproliferative disease of red blood cells). Patients suffering from paroxysmal nocturnal hemoglobinuria (PNH) appear to be especially at risk for Budd-Chiari syndrome, more than other forms of thrombophilia: up to 39% develop venous thromboses and 12% may acquire Budd-Chiari.

A related condition is veno-occlusive disease, which occurs in recipients of bone marrow transplants as a complication of their medication. Although its mechanism is similar, it is not considered a form of Budd-Chiari syndrome.

Other toxicologic causes of veno-occlusive disease include plant & herbal sources of pyrrolizidine alkaloids such as Borage, Boneset, Coltsfoot, T'u-san-chi, Comfrey, Heliotrope (sunflower seeds), Gordolobo, Germander, and Chaparral.

Any obstruction of the venous vasculature of the liver is referred to as Budd-Chiari syndrome, from the venules to the right atrium. This leads to increased portal vein and hepatic sinusoid pressures as the blood flow stagnates. The increased portal pressure causes increased filtration of vascular fluid with the formation of ascites in the abdomen and collateral venous flow through alternative veins leading to esophageal, gastric and rectal varices. Obstruction also causes centrilobular necrosis and peripheral lobule fatty change due to ischemia. If this condition persists chronically what is known as nutmeg liver will develop. Renal failure may occur, perhaps due to the body sensing an “underfill” state and subsequent activation of the renin-angiotensin pathways and excess sodium retention.

When Budd-Chiari syndrome is suspected, measurements are made of liver enzyme levels and other organ markers (creatinine, urea, electrolytes, LDH).

Budd-Chiari Syndrome is most commonly diagnosed using ultrasound studies of the abdomen and retrograde angiography. Ultrasound may show obliteration of hepatic veins, thrombosis or stenosis, spiderweb vessels, large collateral vessels, or a hyperechoic cord replacing a normal vein. Computed tomography (CT) or magnetic resonance imaging (MRI) is sometimes employed although these methods are generally not as sensitive. Liver biopsy is nonspecific but sometimes necessary to differentiate between Budd-Chiari Syndrome and other causes of hepatomegaly and ascites, such as galactosemia or Reye's syndrome.

There are at least three reasons causing Budd-Chiari Syndrome patients being diagnosed and treated. First, there are many sub-types of Budd-Chiari Syndrome. Second, liver structures may be quite different among people. Third, Budd-Chiari Syndrome occurs in 1 out of a million individuals, which is very rare and it is therefore very difficult to study systematically for accumulating experience and for testing innovative and effective way to treat this syndrome.

### SUMMARY OF INVENTION

An objective of the present invention includes a method for treating hepatic area vein occlusion of a patient, including following steps.

Identify a connected position of the inferior vena cava and an accessory hepatic vein, the accessory hepatic vein having occlusion. Insert a guiding wire to the connected position. Break through the occlusion of the accessary hepatic vein with a puncture device carried by the guiding wire. Apply a balloon to enlarge an inner wall of the accessory hepatic vein to keep opening of the accessory hepatic vein.

There are several types for placing guiding wires. For example, the guiding wire may be placed from a jugular vein to an iliac vein of the patient. The guiding wire may be also placed with one end from a jugular vein and the other end via percutaneous transhepatic access path. The guiding wire may also be placed with a portion crossing two hepatic veins. Or, the guiding wire may be placed in concentric through-to-through manner.

The hepatic vein occlusion may be Budd-Chiari Syndrome. Furthermore, a balloon may be used for enlarging the occlusion portion of hepatic veins or inferior vena cava. The balloon may be enlarged for several times and each time with duration less than 1 minute.

The entering path of the puncture device may be under the direction from the superior vena cava to the inferior vena cava. The puncture device may be used for digging and removing occlusion to rebuild a portion of the accessory hepatic vein.

Besides, the type of thrombosis is determined and corresponding treatment is applied to the thrombosis. Fluoroscopy may be used to provide real time images for determining occlusion removing result. A stent may be placed in occlusion portion of the accessory hepatic vein.

The aforementioned method may be applied after a period of time, e.g. after one month of the first treatment.

According to another objective of the present invention, a system is used for treating hepatic area vein occlusion. The system includes a medical image device, a robot, and a control device.

The medical imaging device is used for generating real time images of a patient during interventional treatment. The robot is used to trigger and move devices of the interventional treatment. The control device is used for a physician to see the real time images and to operate the robot to conduct the interventional treatment, wherein the robot is instructed to insert a guiding wire to an accessory hepatic vein with occlusion and to use a puncture device to remove the occlusion.

The control device automatically compares the real time images with database images to estimate the type of occlusion and provide instant suggestion and information to the physician. The control device automatically detects abnormal operation compared with predetermined rules on operating the robot. The control device provides guidelines and suggestions in real time to the physician. The control device performs image processing to combine multiple medical image sources. The multiple medical image sources comprise MRI and X-ray images. A sensor may be carried to the place of occlusion for identifying blocked blood vessel tissue from other hepatic tissue. A guiding message may be provided to the physician to make correct decision or even facilitate correction operation.

### BRIEF DESCRIPTION OF DRAWINGS

FIG. 1 is a diagram illustrating a part of vascular system and organs related to the disclosed embodiments;

FIG. 2 illustrates a guiding wire getting through the heart;

FIG. 3 is a simplified diagram of the first type of guiding wire through-to-through technique;

FIG. 4 is a simplified diagram of the second type of guiding wire through-to-through technique;

FIG. 5 is a simplified diagram of the third type of guiding wire through-to-through technique;

FIG. 6 is a simplified diagram of the fourth type of guiding wire through-to-through technique;

FIG. 7 illustrates a first common way to classify the liver into four segments;

FIG. 8 is a simplified diagram illustrating how several hepatic veins help drain blood in the liver and deliver the blood into the inferior vena cava;

FIG. 9 is a sectional view showing relation of these sections marked with Roma alphabet with three major hepatic veins;

FIG. 10A illustrates a type of BCS;

FIG. 10B illustrates another type of BCS;

FIG. 10C-10G illustrates an operation for opening occlusion of a hepatic accessory vein;

FIG. 11 is a treatment flowchart;

FIG. 12 is the third technique of a preferred embodiment;

FIG. 13 illustrates the guiding wire that carries puncture devices being inserted from the upper side to the down side; and

FIG. 14 illustrates a treatment system.

### DETAILED DESCRIPTION

As mentioned above, Budd-Chiari Syndrome occurs only 1 out of a million, which is rare. But, for more than twenty years of observation, it is found in certain areas of China, Budd-Chiari Syndrome has a relatively high occurrence. Nevertheless, the inventors in the center of these areas of China have taken care of more than 2,000 Budd-Chiari Syndrome patients. The following treatment methods for Budd-Chiari Syndrome, mainly are based on following interventional radiology techniques, with combined cure rates higher than 95%.

Interventional radiology (abbreviated IR or VIR for Vascular and Interventional Radiology, also referred to as Surgical Radiology) is an independent medical specialty, which was a sub-specialty of radiology until recently, that uses minimally invasive image-guided procedures to diagnose and treat diseases in nearly every organ system. The concept behind interventional radiology is to diagnose and treat patients using the least invasive techniques currently available in order to minimize risk to the patient and improve health outcomes.

As the inventors of angioplasty and the catheter-delivered stent, interventional radiologists pioneered modern minimally invasive medicine. Using X-rays, CT, ultrasound, MRI, and other imaging modalities, interventional radiologists obtain images which are then used to direct interventional instruments throughout the body. These procedures are usually performed using needles and narrow tubes called catheters, rather than by making large incisions into the body as in traditional treatment.

Many conditions that once required treatment can now be treated non-surgically by interventional radiologists. By minimizing the physical trauma to the patient, peripheral interventions can reduce infection rates and recovery time, as well as shorten hospital stays.

In the following preferred embodiments, there are five main technologies applied. These technologies may be applied separately or combined under various circumstances and considerations.

Please be also noted that it is found BCS having several different types. It is critical to identify the BCS type of a patient and then applying corresponding treatment to significantly improve treatment effectiveness.

The first technique is referred to as the guiding wire through-to-through technique, which means that a guiding wire used in interventional treatment has its two ends escaped from one or two access entries of a patient. A physician may operate the two ends to effectively and accurately to perform corresponding interventional treatment.

In the preferred embodiment, the guiding wire through-to-through technique is applied on treating Budd-Chiari Syndrome. Therefore, a guiding wire may enter the body of a patient via a jugular vein, an iliac vein, or via skin above the liver of the patient and exit the body of the patient from the same or another access point. At least four ways to implement the guiding wire through-to-through technique are illustrated and explained as follows.

The guiding wire is used for carrying a wider wire or other devices like balloons, stents, radiopaque contrast dye component, etc. to perform interventional treatment like embodiments as explained as follows.

The first type of guiding wire through-to-through technique is to choose a jugular vein and an iliac vein as an entry and an exit points, respectively. A guiding wire is inserted into the jugular vein, via the superior vena cava to enter the right atrium, then to the inferior vena cava, passing the hepatic vein area, continuing to go downwardly until to escape via an iliac vein.

Because two ends of the guiding wires are exposed outside so that a physician may perform the treatment more accurately.

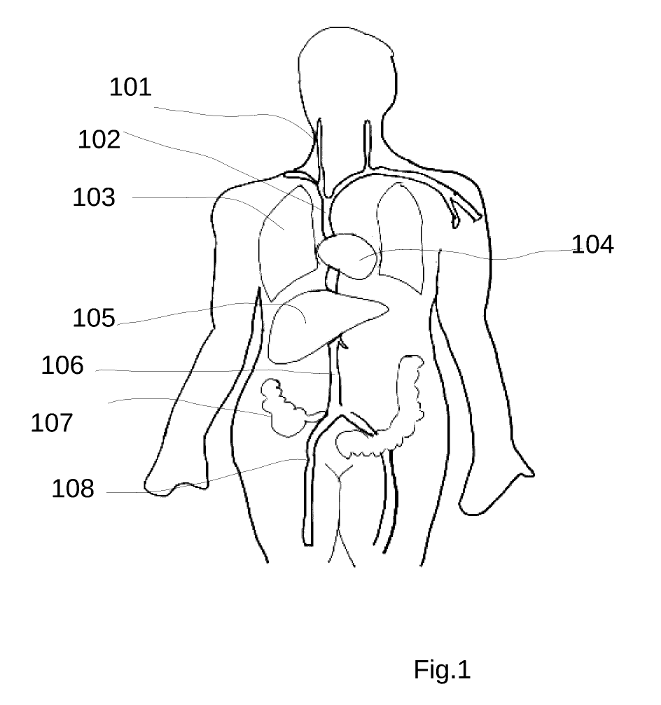

Please refer to FIG. 1, which is a diagram illustrating a part of vascular system and organs related to the disclosed embodiments.

As mentioned above, the heart 104 collects blood from veins and transports the blood to the lung 103 for performing oxygen and carbon dioxide exchange. The jugular vein 101 is connected to the superior vena cava 102 via the subclavian vein to transmit blood into the heart 104. On the other hand, blood from lower body is transmitted by the iliac vein 108, entered the inferior vena cava 106 that connects to the heart 104. In addition, veins from the digestive system 107 transmit blood with nutrient that is sent into the liver 105 via the hepatic portal vein.

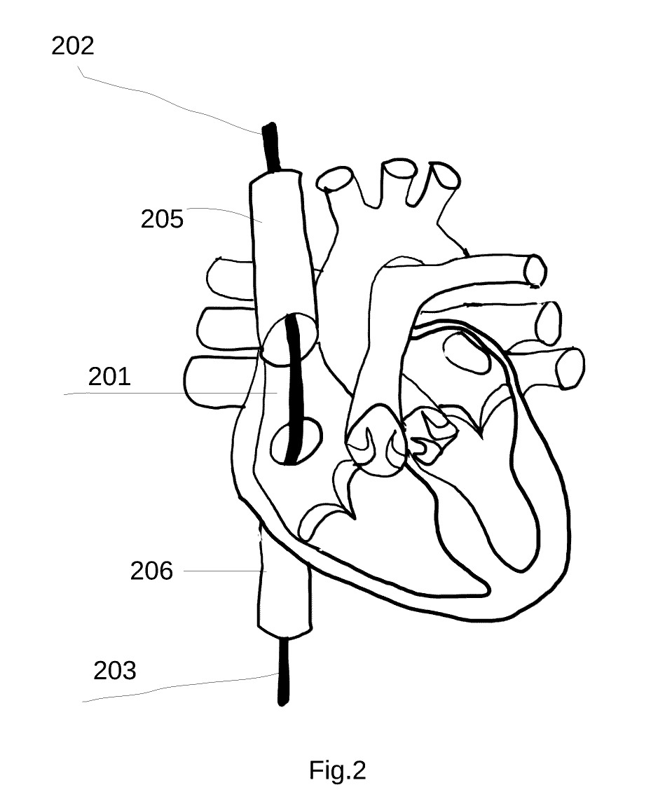

Please refer to FIG. 2, which illustrates how a guiding wire gets through the heart 201. The guiding wire has two ends 202 and 203 extending upwardly and downwardly, respectively. The guiding wire is moving inside the superior vena cava 205 to enter the right atrium 201 and then entering the inferior vena cava 206.

In other words, a guiding wire may move through a channel from an entry point of a jugular vein to an exit point of an iliac vein. During such moving path, the guiding wire meets the liver via several major hepatic veins and some accessory veins.

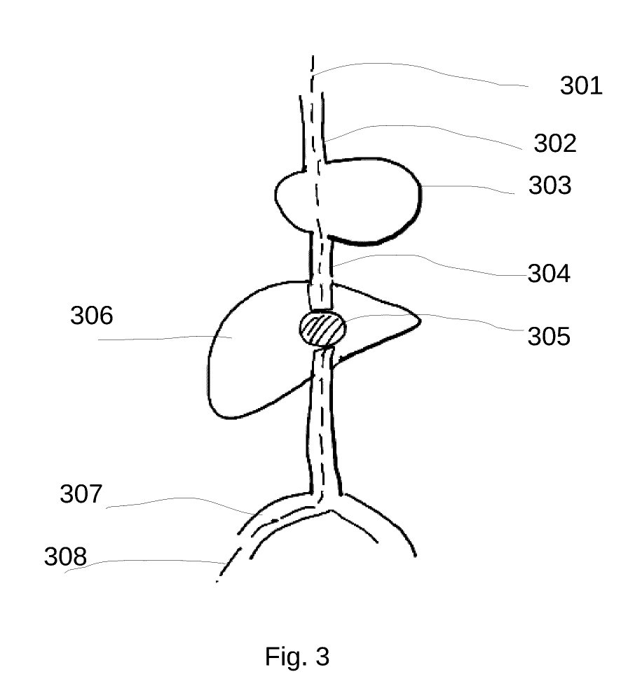

Please refer to FIG. 3, which illustrates a simplified diagram of the first type of guiding wire through-to-through technique. As explained above, in the first type of guiding wire through-to-through technique, a guiding wire has two ends 301, 308 exposed to a physician to operate. The guiding wire enters the superior vena cava 302 into the heart 303. The guiding wire then moves to the inferior vena cava 302 via the right atrium of the heart 303. The guiding wire finally moves downwardly to the iliac vein 307 and exits the body as the second end 308. Please also be noted that there are several veins from the liver 306 entering the inferior vena cava 304.

In this illustrated case, a balloon 305 is put in the inferior vena cava 304 to enlarge the inner wall to solve occlusion. Moreover, because two ends 301, 308 of the guiding wire are exposed outside the patient body, it is easier for a physician to operate more accurately and effectively.

The second type of guiding wire through-to-through technique is a percutaneous transhepatic angioplasty type of intervention. Compared with the first type of guiding wire through-to-through technique, a different end of a guiding wire exits a patient body via skin above the liver of the patient instead of the iliac vein.

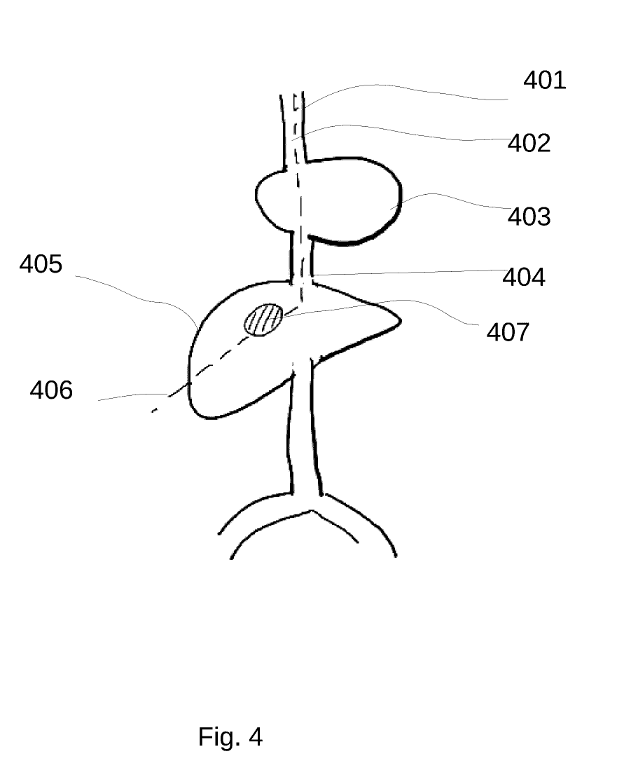

Please refer to FIG. 4, which illustrates a simplified diagram of the second type of guiding wire through-to-through technique. A guiding wire 402 moves through the superior vena cava 401, e.g. via the jugular vein. The guiding wire 402 moves across the heart 403, enters one of hepatic veins connected to the inferior vena cava 404. The guiding wire then goes through the liver 405 and exits the patient body to expose a second end 406. A balloon 407 may be applied to enlarge the blocked portion of a hepatic vein.

The third type of guiding wire through-to-through technique is similar to the first type, but has a portion across hepatic veins.

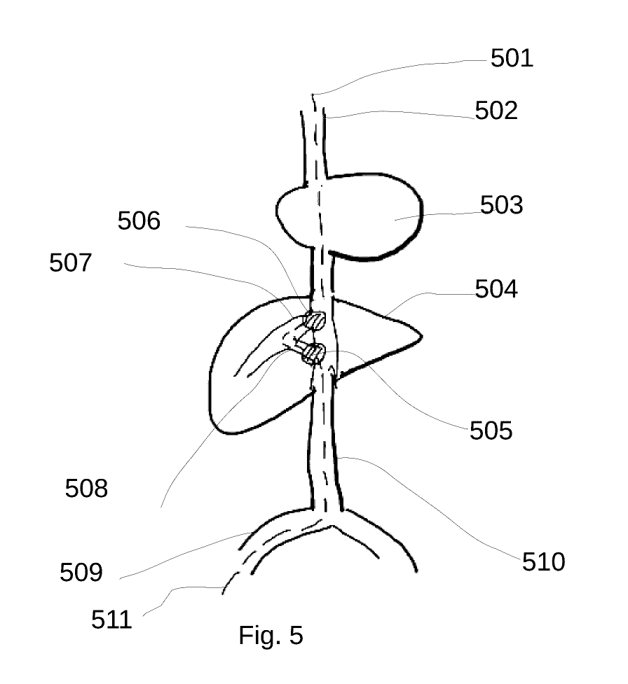

Please refer to FIG. 5, which illustrates a simplified diagram of the third type of guiding wire through-to-through technique. A guiding wire has two ends 501, 511, exposed outside the patient body. One end 501 of the guiding wire enters the superior vena cava 502, and then passes through the heart 503. The other end of the guiding wire moves along the inferior vena cava 510 and exits the patient body via the iliac vein 509. In the moving path, a portion of the guiding wire enters a first hepatic vein 507 from the inferior vena cava 510 into the liver 504 and moves back to the inferior vena cava 510 via a second hepatic vein 508. The first hepatic vein 507 and the second hepatic vein 508 may be selected from the hepatic veins and/or accessory hepatic veins.

Some balloons, e.g. two balloons 505, 506, may be applied at certain positions to enlarge occlusion vein portions.

The fourth type of guiding wire through-to-through technique may be called as the concentric through-to-through. The fourth type is similar to the third type but the guiding wire moves back to the same entry point, e.g. the jugular vein, instead of moving downwardly to the iliac vein to exit the patient body.

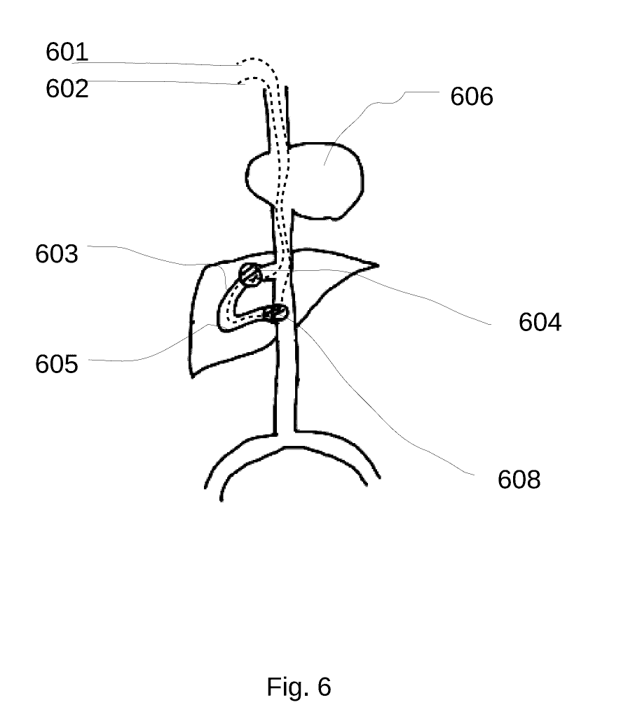

Please refer to FIG. 6, which illustrates a simplified diagram of the fourth type of guiding wire through-to-through technique. As mentioned above, the two ends 601, 602 of a guiding wire are at the same position. The guiding wire moves downwardly via the heart 606 to the inferior vena cava. In the hepatic area of the inferior vena cava, the guiding wire moves into one hepatic vein 603 and leaves via an accessory hepatic vein 605 that connects to the inferior vena cava.

Similarly, some balloons, e.g. the balloons 604, 608, may be applied at certain positions to enlarge occlusion vein portions.

The hepatic vein structure and occlusion are varying among patients and thus different types of guiding wire through-to-through techniques and positions for placing balloons may depend on different circumstances and judgment of physicians. CT, MRI, and other discovering tools may be applied for helping physicians to make decisions.

The second technique is to use one or more accessory hepatic veins (AHV) to drain hepatic blood from the liver into inferior vena cava for decreasing pressure inside the liver and for saving as much hepatic cells to keep function normally as possible.

Before describing the technique in detail, the liver and its vein vascular systems are explained first. The liver is a complicated organ and there are more than one ways to divide the liver into segments.

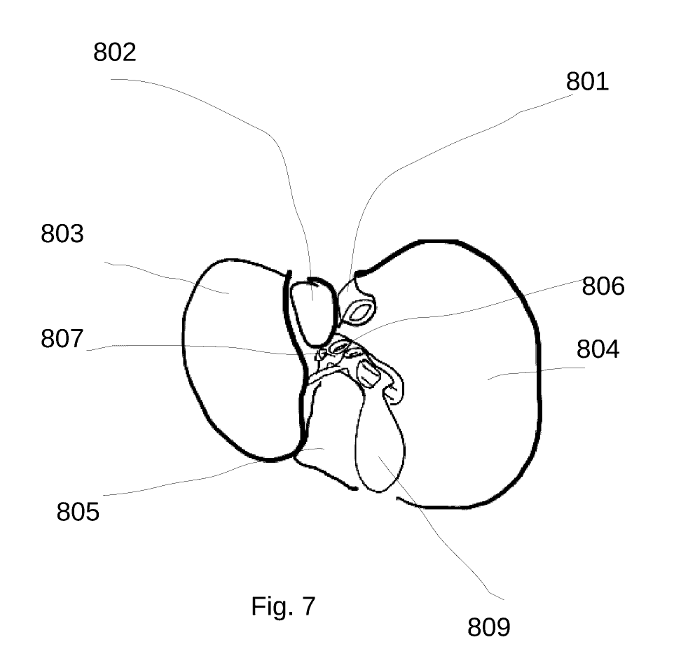

Please refer to FIG. 7, which illustrates a first common way to classify the liver into four segments, i.e. the left lobe 803, the right lobe 804, the caudate lobe 802 and the quadrate lobe 805. The gallbladder 809 is located near the right lobe 804. In addition, the hepatic portal vein 806 is used for collect veins from the digestive system into the liver to process, and the hepatic artery 807 is used for carrying fresh oxygen into liver. The blood are processed and used by the liver cells and then collected into the inferior vena cava 801 via several hepatic veins, to be explained as follows.

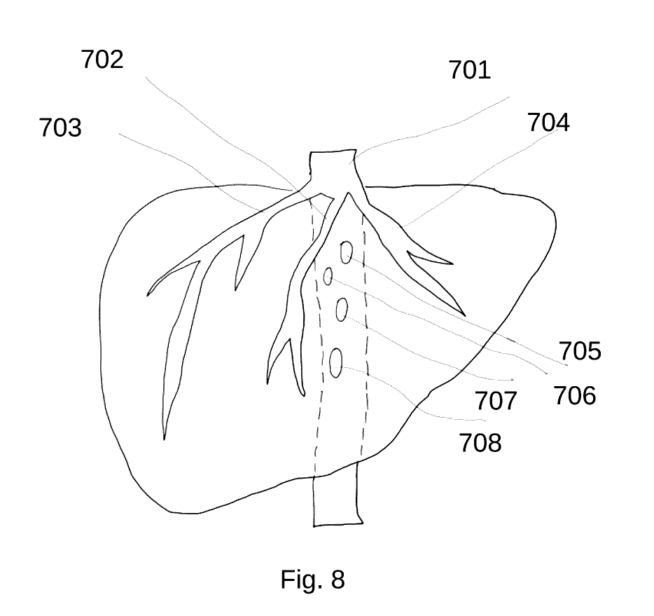

Please refer to FIG. 8, which is a simplified diagram illustrating how several hepatic veins help drain blood in the liver and deliver the blood into the inferior vena cava 701. As illustrated in FIG. 8, there are three major hepatic veins, i.e. the right hepatic vein 703, the middle hepatic vein 702 and the left hepatic vein 704, collected to the inferior vena cava 701. In addition, there are several accessory hepatic veins 705, 706, 707 and 708 connected to the inferior vena cava 701.

Usually, the hepatic veins are classified into two major groups by their locations. On the upper part of liver, the hepatic veins are called major hepatic veins while the hepatic veins on the lower part are called accessory hepatic veins. In addition to accessory hepatic veins, there may be one or more minor hepatic veins also transmitting blood flowing into the inferior vena cava.

In addition to the classification illustrated in FIG. 8, the Couinaud classification is another way to classify the liver into eight sections. Under this classification, each section has its own vascular inflow, outflow and biliary drainage.

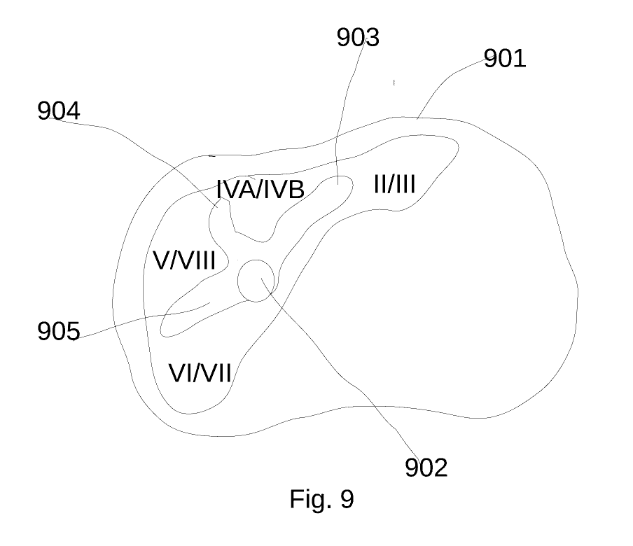

Please refer to FIG. 9, which is a sectional view showing relation of these sections marked with Roma alphabet with three major hepatic veins 903, 904, 905 and the inferior vena cava 902 in a liver 901. In the center of each segment, there is a branch of the portal vein, hepatic artery and bile duct. In the peripheral of each segment, there is a vascular outflow through the hepatic veins. The right hepatic vein divides the right lobe into anterior and posterior segments. The middle hepatic vein divides the liver into right and left lobes (or right and left hemi-liver). This plane runs from the inferior vena cava to the gallbladder fossa. The portal vein divides the liver into upper and lower segments. The left and right portal veins branch superiorly and inferiorly to project into the center of each segment.

Because of this division into self-contained units, each segment can be resected without damaging those remaining. On considering vein occlusion problem, we therefore can treat the liver as multiple units and try to reduce the problem as much as possible. Though in some cases not able to solve the problem completely, certain improvement already helps patients to regain a better life quality.

Please be noted that although FIG. 8 and FIG. 9 illustrate basic liver structure, during clinic research, the actual forking and number of hepatic veins as well as accessary hepatic veins may vary among people. Furthermore, the diameter of these hepatic veins may vary from one another. In the study of “Evaluation of The Variations of The Hepatic Veins” by O Fersia et al. from The Internet Journal of Human Anatomy, 2010 Vol. 2, No. 1, 44.4% of people have seven to eight hepatic veins. But it is also noted that some people may have four to even fifteen hepatic veins. In addition, it is also noted in the study that about 50% of people have only two major hepatic veins, i.e. the left hepatic vein and the right hepatic vein, instead of having all three major hepatic veins.

When any of these hepatic veins have occlusion, blood flow into the inferior vena cava is decreased. Not only corresponding processing of the liver is decreased but also the liver cells may be damaged under long time occlusion.

On the other hand, if more occlusion, even not all, can be reduced, the blood pressure inside the liver as well as number of surviving liver cells may be improved.

In addition to structure variation of the liver among people, the blockage may also vary among people. In the clinical research, the inventors collect at least following different types of occlusion.

The first type is occlusion occurs in the inferior vena cava. Because the inferior vena cava not only helps transmits blood outside the liver but also helps transmit blood from lower body into the heart, such occlusion usually causes serious problem. The aforementioned guiding wire through-to-through technique is very suitable on solving this problem, because the physician have two ends to both perform pull and push operations to open the blockage.

In addition to the first type, there are many different occlusion types. For example, occlusion occurs in a hepatic vein or multiple hepatic veins, or occlusion occurs in both major hepatic veins and accessory hepatic veins.

Under clinic experience, when the hepatic vein and the accessory hepatic vein both have occlusion, and the hepatic vein is a segment occlusion, i.e. with blockage more than a distance, some blocked accessory hepatic veins may have abnormal diameter expansion, e.g. with diameter larger than 8 mm. In such case, usually the liver cells around the blocked major hepatic veins also have cirrhosis, and thus even the major hepatic vein is clear, the consequence might not appear very well. In such case, the inventor finds that the accessory hepatic vein, when its occlusion is removed, the overall occlusion and situation of the patients are significantly improved. However, please be noted that not everyone has accessory hepatic veins. In the 2,000 cases handled by the inventors, about 57% of patients have accessory hepatic veins. If the accessory hepatic veins are blocked, this technique may be applied. Another advantage for using this technique is that the connecting point of an accessory hepatic vein to the inferior vena cava is farther to the heart, about 5 cm, than the connecting point of a major hepatic vein to the heart, about 1 cm. The treatment to open the occlusion in the major hepatic veins may bring much more risk on heart damage than applying treatment to the accessory hepatic veins.

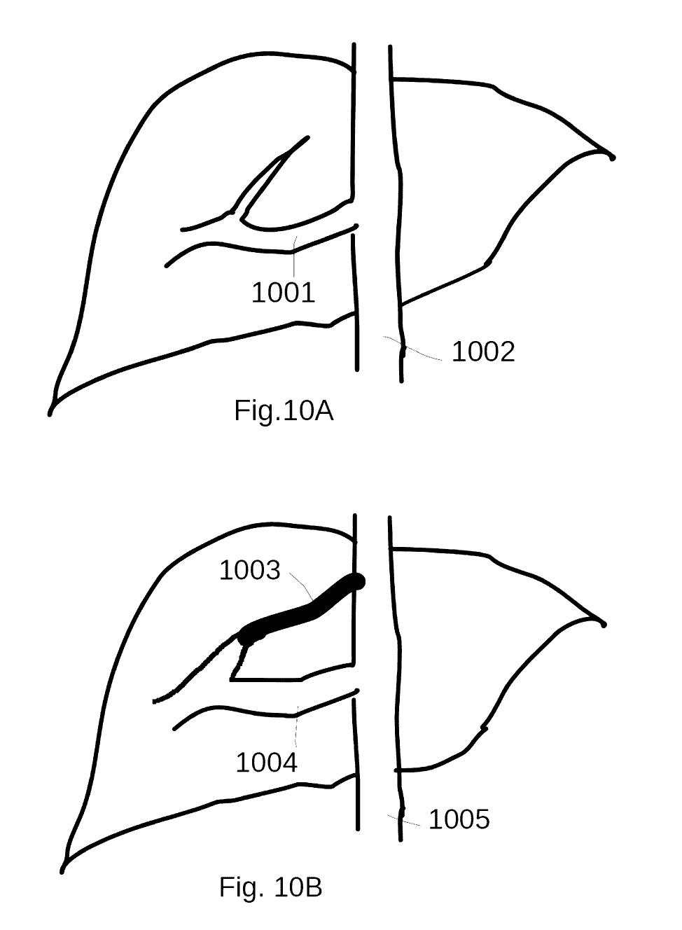

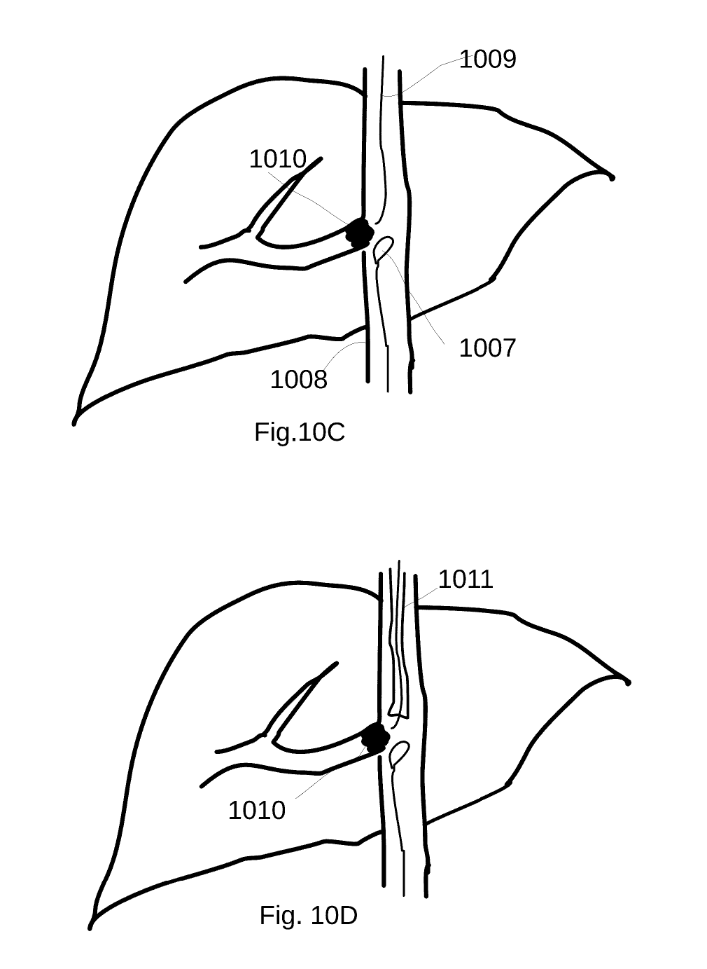

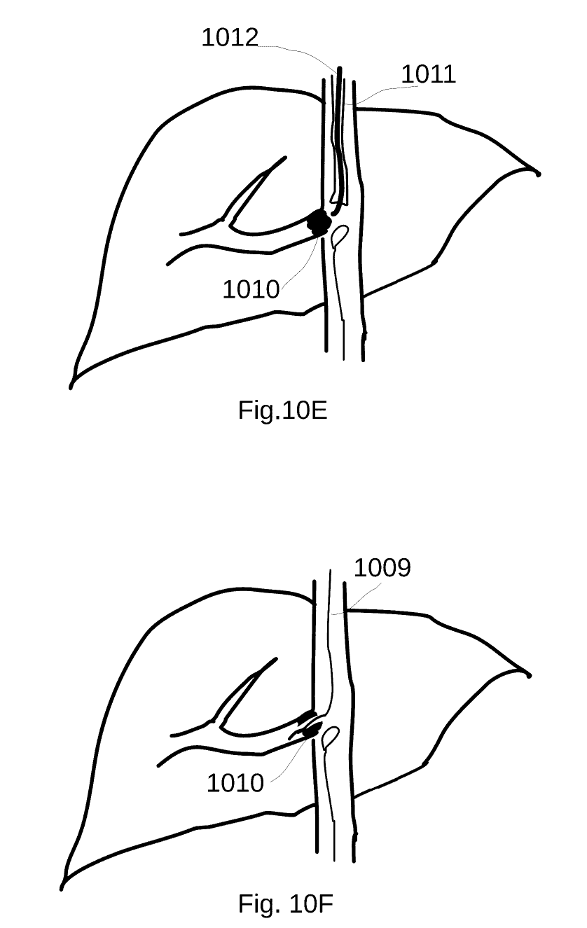

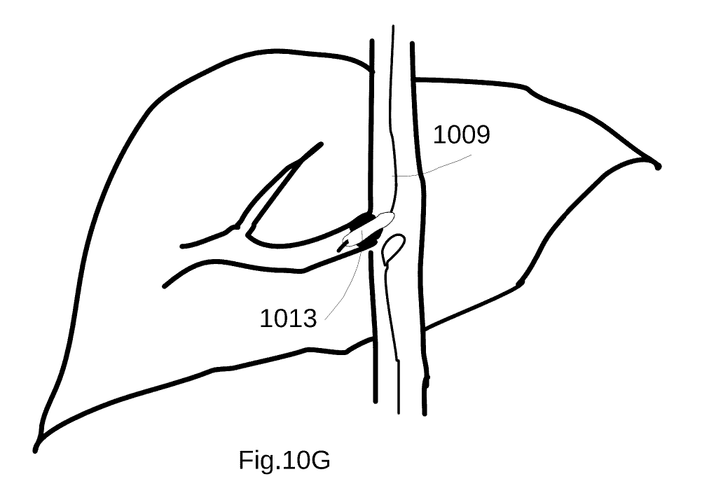

Please refer to FIG. 10A and FIG. 10B, which illustrate two examples of BCS. In FIG. 10A, the major hepatic vein simply cannot be identified. The blood in the liver is flowing out of the liver via an accessory hepatic vein 1001 to the inferior vena cava 1002. In FIG. 10B, a major hepatic vein 1003 is identified but it is completely blocked or looked like a string. In such case, the blood also flows out of the liver via an accessory hepatic vein 1004 to the inferior vena cava 1005.

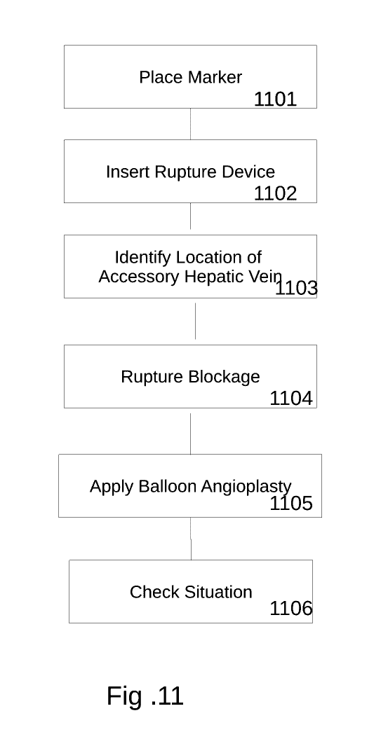

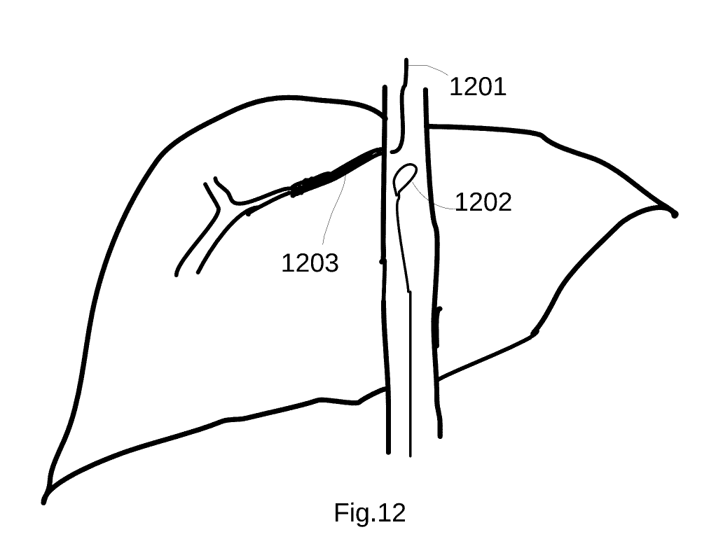

Next, please refer to FIG. 10C to FIG. 10G and FIG. 11. FIG. 10C to FIG. 10G illustrate a treatment to open occlusion of an accessory hepatic vein to decrease the pressure of a liver. In other words, the accessory hepatic vein is used for replacing the function of a corresponding major hepatic vein. FIG. 11 is a flowchart illustrating the treatment steps.

In FIG. 10C, a guiding wire carrying a marker 1007 is inserted from the iliac vein to enter the inferior vena cava 1008 (step 1101). Another guiding wire 1009 is inserted from the jugular vein (step 1102). The entry location of the accessory hepatic vein to the inferior vena cava is identified (step 1103).

In FIG. 10D, a catheter 1011 is inserted via the guiding wire 1009. In FIG. 10E, a puncture device 1012, e.g. a thick wire, is transmitted via the catheter 1011 to the location of blockage 1010. In FIG. 10F, the blockage 1010 is identified and punctured with the puncture device and then the guiding wire 1009 is inserted and getting through the opening of the blockage 1010 (step 1104). In FIG. 10G, a balloon 1013 is carried by the guiding wire 1009 and enlarged to enlarge the inner wall of the accessory hepatic vein (step 1105). The situation is checked (step 1106).

This method is further explained as follows. First, the liver has several segments as explained above. The segment with occlusion has a corresponding major hepatic vein and one or more than one accessory hepatic veins. Usually, the major hepatic vein has larger diameter and is better for solving occlusion. However, in some cases, when the major hepatic vein has segmented occlusion, i.e. with blockage length larger than a distance, it may be difficult or even impossible to get through the major hepatic vein. Under such case, the accessory hepatic vein is selected to replace the channel responsible for draining the blood from the liver to the inferior vena cava.

Second, fluoroscopy, which is an imaging technique that uses X-rays to obtain real-time moving images of the interior of an object, is applied for helping identifying the location of the puncture device, the marker and the situation. In other words, the physician depends on the real time images to determine positions of the device inside the vascular.

Third, radiopaque contrast dye may be applied to determine the flow path for identifying the occlusion position and type.

Fourth, when the blockage portion of occlusion is broken with the puncture device, a balloon is further placed in the place of occlusion to enlarge and/or stabilize the inner wall of the blood vascular. The balloon of Percutaneous Transluminal Angioplasty (PTA) may be kept for one to three minutes. However, because such operation causes serious pain in the patient, in real clinic practice, the balloon of PTA is enlarged for three times, each time lasting for about 20 seconds to 1 minute. Usually, the patients are checked to determine the result of the treatment. Under such operation, it is found about 13% of restenosis rate.

Sixth, stents and/or related medicine may be applied under suitable circumstances.

As mentioned above, some people may not have accessory hepatic veins and the second technique by opening blockage of an accessory hepatic vein cannot be applied. Besides, there are several different types of BCS. For example, the occlusion may occur in both the connecting parts near the inferior vena cava and the occlusion size is not large. In such case, a puncture device like a thick wire may be applied to break the occlusion by creating an opening and then applying a balloon to enlarge the opening. In another type of BCS, the occlusion may be combined with thrombosis. When such case is found, treatment to dissolving thrombosis is applied at the same time. Yet, there is another case when a patient does not have an accessory hepatic vein and all major hepatic veins are blocked. In such case, hepatic vein rebuilding explained as follows may be applied.

The third technique is to rebuild the hepatic vein. In addition to the accessory hepatic vein occlusion reducing as mentioned above or in some cases, accessory hepatic veins not available to perform the treatment as mentioned above, the major hepatic vein under occlusion needs to puncture to get through.

However, the blockage may be serious and the occlusion length may be longer than 1 cm. In such case, a vein rebuilding procedure is applied.

Please refer to FIG. 12, which illustrates the third technique of a preferred embodiment. A marker 1202 is placed in the inferior vena cava and fluoroscopy is used for providing real time image. A membrane puncture device 1201 is inserted slowly following anatomy direction in the hepatic vein 1203. After the puncture device is inserted, a PTA balloon may be further applied to keep the inner wall of the rebuilt hepatic vein. As mentioned above, the puncture device may be carried inside a catheter which is lead firstly via a soft guiding wire. Such procedure prevents unwanted damage to hurt the heart which is quite close to the connection point of the hepatic vein and the inferior vena cava.

Moreover, the balloon technique and parameters as mentioned above for explaining the accessory hepatic vein opening may be applied in this technique.

TIPSS, Transjugular Intrahepatic Portosystemic Shunt or Transjugular Intrahepatic Portosystemic Stent Shunting, refers to an artificial channel within the liver that establishes communication between the inflow portal vein and the outflow hepatic vein and to such treatment. It is used to treat portal hypertension (which is often due to liver cirrhosis), which frequently leads to intestinal bleeding, life-threatening esophageal bleeding (esophageal varices) and the buildup of fluid within the abdomen (ascites).

An interventional radiologist creates the shunt using an image-guided endovascular (via the blood vessels) approach, with the jugular vein as the usual entry site.

The procedure was first described by Josef Rösch in 1969 at Oregon Health and Science University. It was first used in a human patient by Dr. Ronald Colapinto, of the University of Toronto, in 1982, but did not become reproducibly successful until the development of endovascular stents in 1985. In 1988 the first successful TIPS was realized by M. Rössle, G. M. Richter, G. Nöldge and J. Palmaz at the University of Freiburg. The procedure has since become widely accepted as the preferred method for treating portal hypertension that is refractory to medical therapy, replacing the surgical portocaval shunt in that role.

As mentioned above, Budd-Chiari Syndrome means certain occlusion of hepatic area veins. When occlusion occurs, the pressure rises in the hepatic portal vein and adjacent organs. Instead of getting rid of occlusion, TIPSS focuses on decreasing the pressure. By using TIPS, the blood form the digestive system is directed to the vena cava and then delivered to whole body, and the decreased filtration of blood in the liver, which leads to hyperammonemia. The life quality of patients under TIPSS is immediately deteriorated. For example, taking any high protein food immediately causes many syndromes like significant central nervous system (CNS) abnormalities.

The hepatic vein rebuilding technique shows significant advantages compared with TIPSS, the major hepatic vein under occlusion is punctured to get through, which directly bypass the path of blood to release pressure, the techniques help ensure certain function of the liver and thus improve patient life quality significantly.

The fourth technique is the reverse direction membrane puncture technique. As mentioned above, there are various ways to inert angioplasty devices. In the Budd-Chiari Syndrome treatment, it is particularly dangerous because the inferior vena cava and the liver are close to the heart. However, the risk may be significantly decreased by inserting the puncture device in the reverse direction, i.e. the direction from the jugular vein to the inferior vena cava.

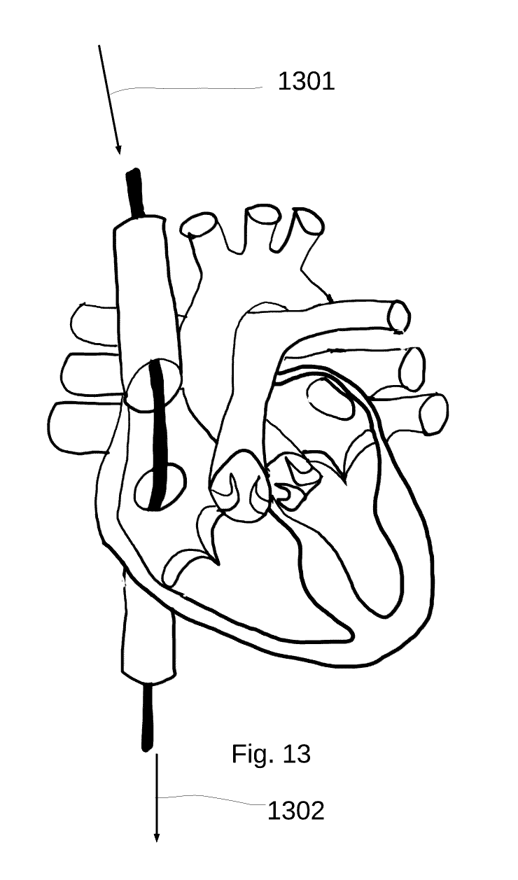

Please refer to FIG. 13, the guiding wire that carries puncture devices is inserted from the upper side 1301 to the down side 1302. In such method, it is clear that the puncture device would not accidently hurt the pericardium. Any damage of the pericardium may cause serious problem. The pericardium is a double-walled sac containing the heart and the roots of the great vessels. The pericardial sac has two layers, a serous layer and a fibrous layer. It encloses the pericardial cavity which contains pericardial fluid.

The pericardium fixes the heart to the mediastinum, gives protection against infection, and provides the lubrication for the heart.

In other words, this technique is to perform the treatment following the direction opposite to the blood flowing in the veins.

The fifth technique is thrombosis handling. Occlusion is usually accompanied with thrombosis. Thrombosis is the formation of a blood clot inside a blood vessel, obstructing the flow of blood through the circulatory system. When a blood vessel is injured, the body uses platelets (thrombocytes) and fibrin to form a blood clot to prevent blood loss. Even when a blood vessel is not injured, blood clots may form in the body under certain conditions. A clot that breaks free and begins to travel around the body is known as an embolus.

When a thrombus is significantly large enough to reduce the blood flow to a tissue, hypoxia (oxygen deprivation) can occur and metabolic products such as lactic acid can accumulate. A larger thrombus causing a much greater obstruction to the blood flow may result in anoxia, the complete deprivation of oxygen and infarction, tissue death. There are also a number of other conditions that can arise according to the location of the thrombus and the organs affected.

Furthermore, if the thrombosis is moved during the angioplasty treatment as mentioned above, the thrombosis may flow along the inferior vena cava to the right atrium, which may causes serious complication. Therefore, to conduct interventional treatment to handle Budd-Chiari Syndrome, thrombosis is a critical issue to be handled.

Under clinical experience, the first thing is to determine the type of the thrombosis. Some thrombosis is strongly attached to the inner wall of veins and does not have immediate risk. However, if thrombosis is determined risky, thrombolytic drugs may be applied and/or guiding tube may be applied to remove thrombosis.

In summary, these techniques bring effective results on hepatic area vein occlusion like Budd-Chiari Syndrome. Different types of occlusion are determined and proper treatments are applied. Unlike traditional aspect of anatomy occlusion, these techniques are based on novel functional occlusion. For example, the accessory hepatic veins, which are not originally regarded as major channels to transmit blood out of the liver, are used to release the occlusion.

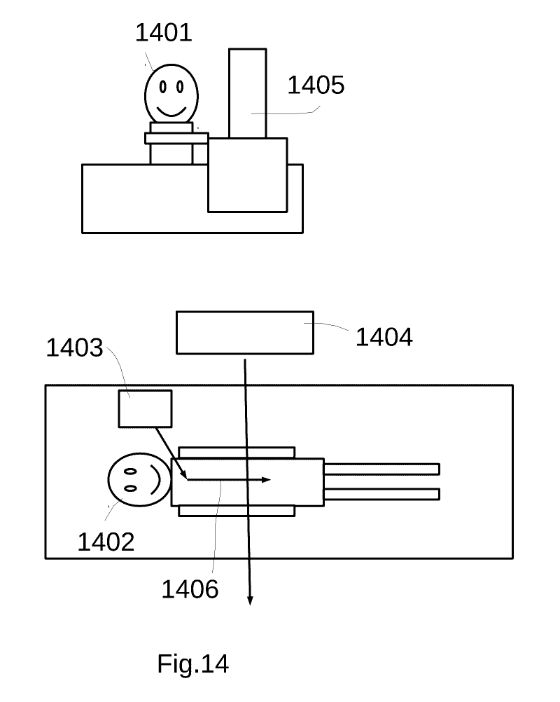

Please refer to FIG. 14, which illustrates a treatment system that may be used for performing the techniques as mentioned above. A physician 1401 performs the treatment before a control device 1405. The control device 1405 provides real time images, e.g. via X-ray technology or other medical image technology during the treatment. A patient 1402 is continuously detected by the medical image device 1404 to capture real time images. A robot 1403 is used to insert guiding wires and/or corresponding devices during interventional treatment. Virtual reality technique may be applied to ensure the treatment to be performed more like by hand, instead of by robot. Because such treatment usually involves with radiology, such configuration reduce risk of the physician in a reasonable level.

Furthermore, the control panel 1405 may compare the real time images with database that collects past experiences. Computer image analysis may be applied to identify the type of occlusion and provides instant intelligent guidance and reminder. This is particularly important for handling rare case like Budd-Chiari, because most physicians do not have experience on such case. These techniques may be further enhanced by advanced sensors and robots.

In addition, a sensor may be applied to detect tissue characteristic to identify vessel blockage from other hepatic tissues so as to help a physician to operate the treatment more accurately particularly in hepatic vein rebuilding. Moreover, images from different techniques may be combined together to provide a physician more information to determine proper treatment in specific cases. For example, CT and MRI images may be combined to provide more information that a single image does not provide.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

1. A method for treating hepatic area vein occlusion of a patient, comprising:
identifying a connected position of the inferior vena cava and an accessory hepatic vein, the accessory hepatic vein having occlusion;
inserting a guiding wire to the connected position;
breaking through the occlusion of the accessary hepatic vein with a puncture device carried by the guiding wire; and
applying a balloon to enlarge an inner wall of the accessory hepatic vein to keep opening of the accessory hepatic vein.

2. The method of claim 1, further comprising placing the guiding wire from a jugular vein to an iliac vein of the patient.

3. The method of claim 1, further comprising placing the guiding wire with one end from a jugular vein and the other end via percutaneous transhepatic access path.

4. The method of claim 1, further comprising placing the guiding wire with a portion crossing two hepatic veins.

5. The method of claim 1, further comprising placing the guiding wire in concentric through-to-through manner.

6. The method of claim 1, wherein the hepatic vein occlusion is Budd-Chiari Syndrome.

7. The method of claim 1, wherein a balloon is used for enlarging the occlusion portion of hepatic veins or inferior vena cava.

8. The method of claim 7, wherein the balloon is enlarged for several times and each time with duration less than 1 minute.

9. The method of claim 1, wherein the entering path of the puncture device is under the direction from the superior vena cava to the inferior vena cava.

10. The method of claim 1, wherein the puncture device is used for digging and removing occlusion to rebuild a portion of the accessory hepatic vein.

11. The method of claim 1, further comprising determining the type of thrombosis and applying corresponding treatment to the thrombosis.

12. The method of claim 1, further comprising using fluoroscopy to provide real time images for determining occlusion removing result.

13. The method of claim 1, further comprising placing a stent in occlusion portion of the accessory hepatic vein.

14. The method of claim 1, further comprising applying the same method after a period of time.

15. A system for treating hepatic area vein occlusion, comprising:
a medical imaging device for generating real time images of a patient during interventional treatment;
a robot to trigger and move devices of the interventional treatment; and
a control device for a physician to see the real time images and to operate the robot to conduct the interventional treatment, wherein the robot is instructed to insert a guiding wire to an accessory hepatic vein with occlusion and to use a puncture device to remove the occlusion.

16. The system of claim 15, wherein the control device automatically compares the real time images with database images to estimate the type of occlusion and provide instant suggestion and information to the physician.

17. The system of claim 15, wherein the control device automatically detects abnormal operation compared with predetermined rules on operating the robot.

18. The system of claim 15, wherein the control device provides guidelines and suggestions in real time to the physician.

19. The system of claim 15, wherein the control device performs image processing to combine multiple medical image sources.

20. The system of claim 19, wherein the multiple medical image sources comprise MRI and X-ray images.
